(19) United States
(12) Patent Application Publication
(10) Pub. No.: US 2008/0090898 A1
(43) Pub. Date: Apr. 17, 2008

(54) Boosting immunity

(75) Inventors: Harold H. Schmitz, Bethesda, MD (US); Catherine L. Kwik-Uribe, Stroudsburg, PA (US)

Correspondence Address: NADA JAIN, P.C., 560 White Plains Road, Suite 460, Tarrytown, NY, 10591, US

(73) Assignee: Mars, Incorporated, McLean, VA (US)

(21) Appl. No.: 11/906,875

(22) Filed: Oct. 4, 2007

Related U.S. Application Data
(60) Provisional application No. 60/849,416, filed on Oct. 4, 2006.

(51) Int. Cl. A61K 31/35

(52) U.S. Cl. 514/456

(57) ABSTRACT

The invention relates to compositions, and methods of use thereof, for boosting immunity, e.g. innate immunity, in a subject in need thereof comprising administering to the subject certain polyphenols such as flavanols, procyanidins, or pharmaceutically acceptable salts or derivatives thereof.

This application claims the benefit, under 35 USC Section 119, of the U.S. Provisional Appl. No. 60/849,416 filed Oct. 4, 2006, the disclosure of which is hereby incorporated herein by reference.

### FIELD OF THE INVENTION

The invention relates to compositions comprising certain polyphenolic compounds and methods of boosting immunity, e.g. boosting innate immunity, in a subject in need thereof comprising administering to the subject certain polyphenolic compounds described herein.

### BACKGROUND OF THE INVENTION

The immune system which is a complex network of tissues, organs, cells and cellular mediators/effectors that protect the body from infection and illness, has two major lines of defense—the innate and adaptive immune responses.

The innate immune system constitutes the first line of defense against pathogens. In addition to providing physical and chemical barriers, the innate immunity consists of several specific immune cells including monocytes/macrophages, natural killer (NK) and polymorphonuclear cells. The innate immune system also plays a critical role in initiating early events in the adaptive immune response.

Applicants have now discovered that the compounds recited herein have a beneficial influence on a variety of early immune responses and are hence effective in boosting immunity, e.g. innate immunity.

### SUMMARY OF THE INVENTION

The invention relates to compositions and products comprising polyphenolic compound(s) described herein and methods of boosting immunity, e.g. innate immunity, comprising administering such compound(s) to a subject in need thereof.

In one aspect, the invention relates to a composition, such as a food (including pet food), a food additive, a dietary supplement, or a pharmaceutical comprising the compound of the invention. Packaged products containing the above-mentioned compositions and a label and/or instructions for use as described herein, e.g., to boost immunity, e.g. innate immunity, are within the scope of the invention.

In another aspect, the invention relates to a method of boosting immunity, e.g., method of boosting innate immunity, comprising administering to a subject in need thereof an effective amount of the compound of the invention.

### BRIEF DESCRIPTION OF THE DRAWINGS

FIG. 1 represents 72 hour proliferation data of purified monocyte cultures from individuals treated with 2.0 uM of (−) Epicatechin, (+) Catechin, 4′-O-methyl-(−)-Epicatechin, 3′-O-methyl-(−)-Epicatechin, A1 Dimer, A2 Dimer, B2 Dimer, B5 Dimer, and the following B-type procyanidins-Trimers, Tetramers, Pentamers, Hexamers, and Heptamers in triplicate wells. Results are reported as a stimulation index (treatment value/by media control value). N=5.

FIG. 2A-B represents: (A) Mean fluorescence intensity of CD 19 stained B cells expressing CD 69 after 16 hour treatment of Long Chain B-type procyanidin Fraction from cocoa (LCFF; black bars) and Short Chain Flavanol and B-type procyanidin Fraction from cocoa (SCFF, gray bars); *indicates significant differences (p<0.05) between cocoa treatment and media control (white bar). (B) Percent of CD 19 stained B cells expressing CD 69 after a 16 hour treatment with LCFF (black bars) and SCFF (gray bars); *indicates significant differences (p<0.05) between cocoa treatment and media control (white bar).

FIG. 3A-B represents: (A) Mean fluorescence intensity of CD 19 stained B cells expressing CD 83 after a 16 hour treatment with LCFF (black bars) and SCFF (gray bars); *indicate significant differences (p<0.05) between cocoa treatment and media control (white bar). (B) Percent of CD 19 stained B cells expressing CD 83 after a 16 hour treatment with LCFF (black bars) and SCFF (gray bars); *indicate significant differences (p<0.05) between cocoa treatment and media control (white bar).

### DETAILED DESCRIPTION

All patents, patent applications and references cited in this application are hereby incorporated herein by reference. In case of any inconsistency, the present disclosure governs.

The invention relates to compositions and products comprising polyphenolic compound(s) described herein and methods of boosting immunity, e.g. innate immunity, comprising administering such compound(s) to a subject in need thereof. The polyphenolic compounds for use in the present invention include certain flavanols (flavan-3-ols), procyanidins (B-type and A-type), or pharmaceutically acceptable salts or derivatives thereof. Such compounds, when of natural origin, may be included in the composition as a plant component such as a cocoa component, for example cocoa nibs or fragments thereof, chocolate liquor, partially and fully-defatted cocoa solids, cocoa extract or fraction thereof, nut skins, peanuts, peanut components e.g., peanut skins, almonds, almond components e.g., almond skins, walnuts, plums (including skin), and cranberries.

As used herein, the term “flavanol” or “flavan-3-ol” refers to a monomer of the following formula:

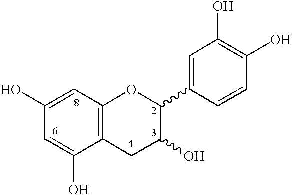

The term “procyanidin” refers to an oligomeric compound composed of monomeric units of the formula shown above and depending on linkages between monomeric units may be a B-type or an A-type procyanidin.

The term “cocoa component” refers to a component derived from cocoa bean, e.g., cocoa nibs and fragments thereof, chocolate liquor, partially and fully-defatted cocoa solids (e.g., cake or powder), flavanol and/or procyanidin-containing cocoa extract or fraction thereof.

In certain embodiments, the present invention relates to a flavanol (e.g., (−)-epicatechin and (+)-catechin), and a composition comprising an effective amount of the flavanol (e.g., (−)-epicatechin and (+)-catechin), or a pharmaceutically acceptable salt or derivative thereof (including oxidation products, esters, methylated derivatives and glucuronidated derivatives, wherein (in certain embodiments) the flavanol derivative is not a gallated derivative). The derivatives may be prepared as described below.

In other embodiments, the present invention relates to a compound, and a composition comprising an effective amount of the compound, having the following formula Aₙ, or a pharmaceutically acceptable salt or derivative thereof (including oxidation products, esters, methylated derivatives and glucuronidated derivatives): 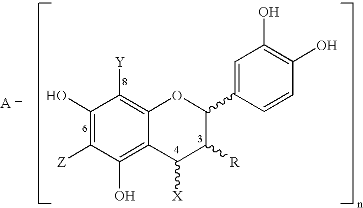
wherein

n is an integer from 2 to 18;

R and X each have either α or β, stereochemistry;

R is OH or O-sugar;

the substituents of C-4, C-6 and C-8 are X, Z and Y, respectively, and bonding of monomeric units occurs at C-4, C-6 or C-8;

when any C-4, C-6 or C-8 is not bonded to another monomeric unit, X, Y and Z independently are hydrogen or a sugar; and

the sugar is optionally substituted with a phenolic moiety at any position, for instance, via an ester bond.

Monomeric units in the formula Aₙ may be bonded via 4→6α; 4→6β; 4→8α; and/or 4→8β linkages. The sugar is preferably a monosaccharide or a di-saccharide. The sugar may be selected from the group consisting of glucose, galactose, rhamnose, xylose, and arabinose. The phenolic moiety may be selected from the group consisting of caffeic, cinnamic, coumaric, ferulic, gallic, hydroxybenzoic and sinapic acids. Procyanidin derivatives may include esters such as the gallate esters; compounds derivatized with a saccharide moiety such as mono- or di-saccharide moiety (e.g., β-D-glucose), glucuronidated (β-D-glucuronide) and methylated derivatives, and oxidation products. In some embodiments, ester derivatives are other than esters with gallic acid. Oxidation products may be prepared as disclosed in U.S. Pat. No. 5,554,645, the relevant portions of which are incorporated herein by reference. Esters, for example esters with gallic acid, may be prepared using known esterification reactions, and for example as described in U.S. Pat. No. 6,420,572, the disclosure of which is hereby incorporated herein by reference. Methylated derivatives, such as 3′-O-methyl-, 4′-O-methyl-, and 3′-O, 4′-O-dimethyl-derivatives may be prepared, for example, as described in the examples and/or in Cren-Olive et al., 2002, J. Chem. Soc. Perkin Trans. 1, 821-830, and Donovan et al., Journal of Chromatography B, 726 (1999) 277-283, the disclosures of which are hereby incorporated herein by reference. Glucuronidated products may be prepared as described in Yu et al, “A novel and effective procedure for the preparation of glucuronides,” Organic Letters, 2(16) (2000) 2539-41, and as in Spencer et al, “Contrasting influences of glucuronidation and O-methylation of epicatechin on hydrogen peroxide-induced cell death in neurons and fibroblasts,” Free Radical Biology and Medicine 31(9) (2001) 1139-46, hereby incorporated herein by reference. Glucuronidation may take place at the 7, 5 and/or 3′ position(s). Examples of glucuronidated products include 4′-O-methyl-epicatechin-O-β-D-glucuronide (e.g., 4′-O-methyl-epicatechin-7-O-β-D-glucuronide), 3′-O-methyl-epicatechin-O-β-D-glucuronide (e.g., 3′-O-methyl-epicatechin-5/7-O-β-D-glucuronides), and epicatechin-O -β-D-glucuronide (e.g., epicatechin-7-O-β-D-glucuronide). It should be noted that this disclosure applies to all formulas recited herein.

In another embodiment, the invention relates to a compound, and the composition comprising an effective amount the compound having the formula Aₙ, or a pharmaceutically acceptable salt or derivative thereof (including oxidation products, esters, methylated derivatives and glucuronidated derivatives), 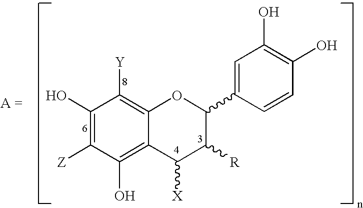
wherein

n is an integer from 2 to 18;

R and X each have either α or β stereochemistry;

R is OH;

the substituents of C-4, C-6 and C-8 are X, Z and Y, respectively, and bonding of monomeric units occurs at C-4, C-6 and C-8; and

when any C-4, C-6 or C-8 is not bonded to another monomeric unit, X, Y and Z are hydrogen.

Examples of the compounds useful for the products and in the methods of the invention include any compounds of the formula Aₙ described herein, wherein the integer n is 3 to 18; 2 to 12; 3 to 12; 2 to 5; 4 to 12; 5 to 12; 4 to 10; or 5 to 10. In some embodiments, the integer n is 2 to 4, for example 2 or 3.

In further embodiments, the present invention relates to a compound, and a composition comprising an effective amount of the compound (A-type procyanidin) which is an oligomer composed of n monomeric, flavan-3-ol units, which flavan-3-ol has the following formula: 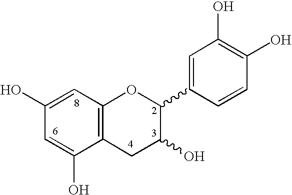
wherein
(i) the monomeric units are connected via interflavan linkages 4→6 and/or 4→8;
(ii) at least two of the monomeric units are additionally linked by an A-type interflavan linkage (4→8; 2→O→7) or (4→6; 2→O→7); and
(iii) n is 2 to 12;
or a pharmaceutically acceptable salt or derivative thereof.

It will be understood by a person of skill in the art that one of the two flavanol units linked by the A-type interflavanoid linkage must comprise two bonds at the 2- and 4-positions. Both of these have either a or C stereochemistry, i.e., the bonds are either 2α, 4α or 2β, 4β. These bonds connect to the 6- and 7-O-positions, or the 8- and 7-O-positions of the second flavanol unit linked by the A-type interflavan linkage. In constituent flavanol units of the oligomer which do not comprise A-type interflavan linkages at positions C-2 and C-4, the linkage at position C-4 can have either alpha or beta stereochemistry. The OH group at position C-3 of flavanol units has either alpha or beta stereochemistry. Flavan-3-ol (monomeric) units may be (+)-catechin or epicatechin.

An A-type procyanidin as defined above may be derivatized, for instance esterified, at one or more of the OH groups on one or more of the constituent flavan-3-ol units. A given flavan-3-ol unit may thus comprise one or more ester groups, e.g., gallate ester group(s), at one or more of the 3-, 5-, 7-, 3′- and 4′-ring positions. It may in particular be a mono-, di-, tri-, tetra- or penta-gallated unit.

Examples of the compounds useful for products, and in the methods of the present invention, include the compounds wherein the integer n is 3 to 12; 4 to 12; 5 to 12; 4 to 10; or 5 to 10. In some embodiments, n is 2 to 4, or 2 to 5, for example n is 2 or 3.

In one embodiment, the invention relates to epicatechin-(4β→8; 2β→O→7)-catechin (i.e., A1 dimer), or a pharmaceutically acceptable salt or derivative thereof, which A1 dimer has the following formula:

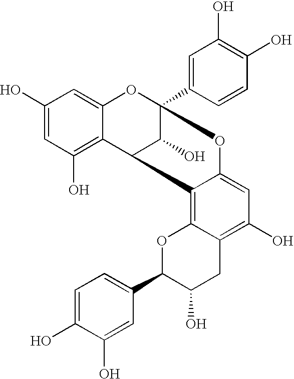

In another embodiment, the invention relates to epicatechin-(4β→8; 2β→O→7)-epicatechin (i.e., A2 dimer), or a pharmaceutically acceptable salt or derivative thereof, which A2 has the following formula: 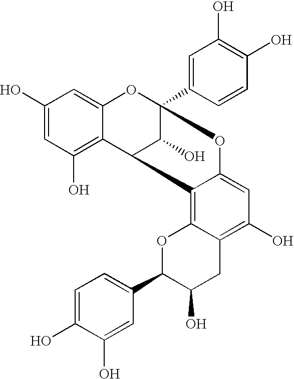
Methods of Use

The invention relates to methods of boosting immunity, e.g. boosting innate immunity, in a subject in need thereof.

As used herein, “boosting immunity” refers to stimulating or promoting an immune status, function and/or response. “Boosting innate immunity” refers to stimulating or promoting innate immunity, which innate immunity is as defined by a standard immunology textbook, e.g. a non-specific part of immune system that defends an organism against an invader in non-specific form regardless of what the invader is. A person of skill in the art would appreciate that boosting innate immunity may also encompass modulating early adaptive immune responses since innate immunity plays a role in initiating early steps of the adaptive immune response.

A “subject in need of boosting innate immunity” is a subject having an increased susceptibility to an invader, e.g. an infectious agent or an environmental toxin, or a subject having an increased risk of and/or exposure to an invader, e.g. an infectious agent or an environmental toxin. Thus, as used herein, “the subject in need thereof” is not a subject having a healthy immune system and a normal or average risk of exposure to an invader. For example, the term “the subject in need thereof” does not include subjects having a healthy immune system and exposed to seasonal viral and/or bacterial agents; the term does include subjects with a healthy immune system who have a higher than average or increased risk of exposure to an invader (e.g. hospital personnel, subjects exposed to environmental toxins). Such a subject may be a human or a veterinary animal.

The term “veterinary animal” refers to any animal cared for, or attended to by, a veterinarian, and includes companion (pet) animals and livestock animals, for example a cat, a dog and a horse.

Examples of subjects having an increased susceptibility to an invader are immunocompromised subjects, i.e., subjects having an impaired immune system usually due to a disease (genetic or acquired), malnutrition or immunosuppressive therapy (e.g., corticosteroid therapy). An immunocompromised subject may be suffering from an immunodeficiency disease, e.g. AIDS, primary immunodeficiency, and/or be a subject vulnerable to opportunistic infections (caused by organisms that usually do not affect a healthy immune system). As used herein, the term “immunocompromised subject” does not include subject(s) suffering from an autoimmune disease; due to their underlying condition, such subjects are not intended for boosting immunity according to the present invention. Subjects suffering from malnutrition are for example those consuming an unbalanced diet e.g., diet lacking in vitamins and minerals or subjects with poor eating habits e.g., anorexic and bulimic subjects. Additional examples of subjects with increased susceptibility to invaders are subjects undergoing prolonged drug treatments e.g., treatment with antibiotics.

Examples of subjects having an increased risk of and/or exposure to an invader, are those subjected to invasive procedures (e.g., surgery, organ/tissue transplant recipients) and devices (e.g., catheters, drainage tubes); persons with occupational hazards e.g. hospital personnel; subjects exposed to certain environmental toxins e.g., naturally-occurring toxins, pollutants; subjects at risk of hospital-acquired infections e.g., particularly those in intensive care units.

In certain embodiments, the present invention provides a method of boosting immunity, e.g. boosting innate immunity, comprising administering to a human or a veterinary animal in need thereof an effective amount of a flavanol of the above formula, such as epicatechin or catechin (e.g., (−)-epicatechin or (+)-catechin), or a pharmaceutically acceptable salt or derivative thereof (including oxidation products, esters, methylated derivatives and glucuronidated derivatives). In certain embodiments, the flavanol derivative is not a gallated derivative.

In other embodiments, the invention provides a method of boosting immunity, e.g. boosting innate immunity, comprising administering, to a human or a veterinary animal, an effective amount of a compound having the following formula Aₙ, or a pharmaceutically acceptable salt or derivative thereof (including oxidation products, esters, methylated derivatives and glucuronidated derivatives): 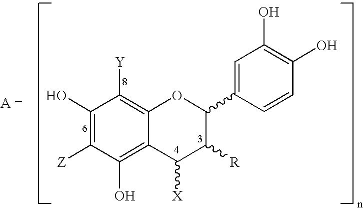
wherein

n is an integer from 2 to 18;

R and X each have either α or β stereochemistry;

R is OH or O-sugar;

the substituents of C-4, C-6 and C-8 are X, Z and Y, respectively, and bonding of monomeric units occurs at C-4, C-6 or C-8;

when any C-4, C-6 or C-8 is not bonded to another monomeric unit, X, Y and Z independently are hydrogen or a sugar; and

the sugar is optionally substituted with a phenolic moiety at any position, for instance, via an ester bond.

For example, the above method may involve use of a compound Aₙ, or a pharmaceutically acceptable salt or derivative thereof (including oxidation products, esters, methylated derivatives and glucuronidated derivatives), wherein R is OH, and when any C-4, C-6 or C-8 is not bonded to another monomeric unit, X, Y and Z are hydrogen. Examples of suitable sugars are as described above. Examples of phenolic moieties are as described above. Examples of derivatives are as described above.

In certain embodiments, the invention provides a method of boosting immunity, e.g. boosting innate immunity, comprising administering to a human or a veterinary animal in need thereof, an effective amount of a compound having the formula Aₙ, or a pharmaceutically acceptable salt or derivative thereof (including oxidation products, esters, methylated derivatives and glucuronidated derivatives): 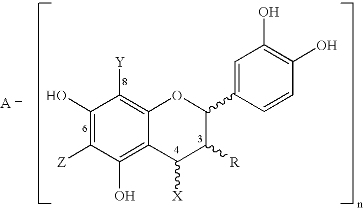
wherein

n is an integer from 2 to 18;

R and X each have either α or β stereochemistry;

R is OH;

the substituents of C-4, C-6 and C-8 are X, Z and Y, respectively, and bonding of monomeric units occurs at C-4, C-6 and C-8; and

when any C-4, C-6 or C-8 is not bonded to another monomeric unit, X, Y and Z are hydrogen.

In further embodiments, the invention provides a method of boosting immunity, e.g. boosting innate immunity, comprising administering to a human or a veterinary animal in need thereof, an effective amount of a compound which is an oligomer composed of n monomeric, flavan-3-ol units, which flavan-3-ol has the following formula: 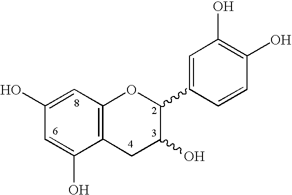
wherein
(i) the monomeric units are connected via interflavan linkages 4→6 and/or 4→8;
(ii) at least two of the monomeric units are additionally linked by an A-type interflavan linkage (4→8; 2→O→7) or (4→6; 2→O→7); and
(iii) n is 2 to 12;
or a pharmaceutically acceptable salt or derivative thereof.

Examples of the compounds useful for the products and in the methods of the invention include the compounds described herein wherein the integer n is 3 to 18; 2 to 12; 3 to 12; 2 to 5; 4 to 12; 5 to 12; 4 to 10; or 5 to 10. In some embodiments, the integer n is 2 to 4, for example 2 or 3. This disclosure applies to any compound of formula An or A-type procyanidin described herein. Examples of A-type procyanidins are A1 and A2 dimer.

The present compounds may be administered in isolated and purified or substantially pure form or as a plant component e.g., plant extract or synthetically prepared. Regarding B-type procyanidins, compounds may be administered as a cocoa component, for example cocoa nibs or fragments thereof, chocolate liquor, partially and fully-defatted cocoa solids (e.g., cocoa powder), cocoa extract or fraction thereof, or may be added independently of cocoa components. The cocoa component may be prepared such that the content of cocoa polyphenols (CP) is preserved. Regarding A-type procyanidins, they may be obtained from natural sources (including plant components), non-limiting examples of which are peanuts, peanut components e.g., peanut skin, almonds, almond components e.g., almond skin, nut skins, plums (including skin), and cranberries.

In some embodiments, the present compounds may be administered in combination with other immune boosting agents. Examples of immune boosting agents are interferon (e.g., Roferon-A®, Intron® A, Infergen®), interleukin (e.g., Proleukin®), nutritional supplements (e.g., vitamin E, vitamin C, vitamin A, vitamin B12), amino acids/peptides/proteins (e.g., glutamine, arginine, glutathione, whey protein), omega-3 fatty acids, zinc, and beta-glucan (a polysaccharide, e.g., beta-1,3-glucan).

Thus, the following uses are within the scope of the invention. Use of a flavanol or a pharmaceutically acceptable salt or derivative thereof (including oxidation products, esters, methylated derivatives and glucuronidated derivatives, wherein (in some embodiments) the derivative is not a gallated derivative), as defined above, in the manufacture of a medicament, food, nutraceutical or dietary supplement for boosting immunity, e.g. innate immunity, in a subject in need thereof. Use of a compound of formula Aₙ, or a pharmaceutically acceptable salt or derivative thereof (including oxidation products, esters, methylated derivatives and glucuronidated derivatives), as defined herein, in the manufacture of a medicament, food, nutraceutical or dietary supplement for boosting immunity, e.g. innate immunity, in a subject in need thereof. Use of an A-type procyanidin, or a pharmaceutically acceptable salt or derivative thereof (including oxidation products, esters, methylated derivatives and glucuronidated derivatives), as defined herein, in the manufacture of a medicament, food, nutraceutical or dietary supplement for boosting immunity, e.g. innate immunity, in a subject in need thereof.

The effective amount may be determined by a person of skill in the art using the guidance provided herein and general knowledge in the art, for example by taking into consideration factors such as administered dose, matrix, frequency of dosing, route of administration, etc. For example, the effective amount may be such as to achieve a physiologically relevant concentration in the body of a mammal. Such a physiologically relevant concentration may be at least 20 nanomolar (nM), preferably at least about 100 nM, and more preferably at least about 500 nM, for example in the blood of the subject, which may be achieved via administration of a single compound or a mixture of compounds described herein. In one embodiment, at least about one micromole in the blood of the mammal, such as a human, is achieved.

The effective amount may be achieved by administration of a single compound or a mixture of compounds described herein. The compounds defined herein, may be administered at from about 35 mg/day, 40 mg/day or 50 mg/day (e.g., to about 1000 mg/day), or from about 75 mg/day (e.g., to about 1000 mg/day), or from about 100-150 mg/day (e.g., to about 900 mg/day), or from about 300 mg/day (e.g., to about 500 mg/day). However, amounts higher than exemplified above may be used since the upper end of the amount range is not a limiting factor. The amounts may be measured as described in Adamson, G. E. et al., J. Ag. Food Chem.; 1999; 47 (10) 4184-4188.

A person of skill in the art will be able to assess the suitable mode of administration of the compounds of the invention e.g., orally, sublingually, bucally, nasally, rectally, by injection, intravenously, parenterally and topically. For example, for boosting immunity, when administered orally, the inventive compounds need not be absorbed into the blood stream as the residence time in the gut/intestinal cells may be sufficient to promote an immune response at the gut level via the gut-associated immune system, e.g. gut-associated lymphoid tissue (GALT).

The administration may be continued as a regimen, i.e., for an effective period of time, e.g., daily, monthly, bimonthly, biannually, annually, or in some other regimen, as determined by the skilled medical practitioner for such time as is necessary. The administration may be continued for at least a period of time required to achieve improvement in a subject recited above. The composition may be administered daily, preferably two or three times a day, for example, morning and evening to maintain the levels of the effective compounds in the body of the mammal. To obtain the most beneficial results, the composition may be administered for at least 7 days, or at least 14 days, or at least 30 days, or at least 45 days, or at least 60 days, or at least 90 days. These regimens may be repeated periodically as needed. The composition may also be beneficial when administered acutely with effects being observable within hours or days, for e.g., with oral administration, or more rapidly with intravenous administration.

Compositions and Formulations

The compounds of the invention may be administered as a food (including pet food), a food additive, or a dietary supplement, or a pharmaceutical.

As used herein, “food” is a material containing protein, carbohydrate and/or fat, which is used in the body of an organism to sustain growth, repair and vital processes and to furnish energy. Foods may also contain supplementary substances, for example, minerals, vitamins and condiments. See Merriam-Webster's Collegiate Dictionary, 10th Edition, 1993. The term food includes a beverage adapted for human or animal consumption. As used herein a “food additive” is as defined by the FDA in 21 C.F.R. 170.3(e)(1) and includes direct and indirect additives. As used herein, a “dietary supplement” is a product (other than tobacco) that is intended to supplement the diet that bears or contains the one or more of the following dietary ingredients: a vitamin, a mineral, an herb or other botanical, an amino acid, a dietary substance for use by man to supplement the diet by increasing the total daily intake, or a concentrate, metabolite, constituent, extract or combination of these ingredients. As used herein, a “pharmaceutical” is a medicinal drug. See Merriam-Webster's Collegiate Dictionary, 10th Edition, 1993. A pharmaceutical may also be referred to as a medicament. The above compositions may be prepared as is known in the art.

The compositions may contain a carrier, a diluent, or an excipient. Depending on the intended use, the carrier, diluent, or excipient may be chosen to be suitable for human or veterinary use, food, additive, dietary supplement or pharmaceutical use. The composition may optionally contain an additional immunity boosting agent. Also depending on use, a person of skill in the art may select the degree of purity of the compound of the invention. For example, when used to prepare pharmaceutical dosage forms, the compound should be as pure as commercially possible, while when preparing food, additive, or supplement, less pure or mixtures of compounds (e.g., plant extracts) may be used.

The compound of the invention may be “isolated and purified,” i.e., it may be separated from compounds with which it naturally occurs (e.g., when the compound is of natural origin), or it may be synthetically prepared, in either case such that the level of contaminating compounds and/or impurities does not significantly contribute to, or detract from, the effectiveness of the compound. For example, an “isolated and purified B2 dimer” is separated from B5 dimer, with which it may occur in nature (e.g., in cocoa bean), to the extent achievable by the available commercially viable purification and separation techniques. Such compounds are particularly suitable for pharmaceutical applications.

The compound may also be less pure, i.e., “substantially pure,” i.e., it may possess the highest degree of homogeneity achievable by available purification, separation and/or synthesis technology but need not be separated from the like compounds. As used herein, “the like compounds” are the compounds having the same degree of polymerization. For example, a “substantially pure dimer” refers to a mixture of dimers (e.g., B2 and B5, as it would occur in a cocoa extract fraction). While less suitable for pharmaceutical applications, such “substantially pure” compounds may be utilized for food, food additive and dietary supplement applications.

In some embodiments, the compound of the invention is at least 80% pure, at least 85% pure, at least 90% pure, at least 95% pure, at least 98% pure, or at least 99% pure. Such compounds are particularly suitable for pharmaceutical applications.

Pharmaceuticals containing the inventive compounds, optionally in combination with another immune-boosting agent, may be administered in a variety of ways such as orally, sublingually, bucally, nasally, rectally, by injection, intravenously, parenterally and topically. As used herein, “oral administration” includes administration by the mouth and includes sublingual and bucal administrations. A person of skill in the art will be able to determine a suitable mode of administration to maximize the delivery of the compounds of the invention. Thus, dosage forms adapted for each type of administration by mouth are within the scope of the invention and include solid, liquid and semi-solid dosage forms, such as tablets, capsules, gelatin capsules (gelcaps), bulk or unit dose powders or granules, emulsions, suspensions, pastes, or jellies. Sustained-release dosage forms are also within the scope of the invention. Suitable pharmaceutically acceptable carriers, diluents, or excipients are generally known in the art and can be determined readily by a person skilled in the art. The tablet, for example, may comprise an effective amount of the compound of the invention and optionally a carrier, such as sorbitol, lactose, cellulose, or dicalcium phosphate.

The foods comprising the compounds described herein and optionally another immune boosting agent may be adapted for human or veterinary use, and include pet foods. The food may be other than a confectionery, for example, a beverage (e.g., cocoa flavored beverage). A confectionery such as a standard of identity (SOI) and non-SOI chocolate, such as milk, sweet and semi-sweet chocolate including dark chocolate, low fat chocolate and a candy which may be a chocolate covered candy are also within the scope of the invention. Other examples include a baked product (e.g., brownie, baked snack, cookie, biscuit), a condiment, a granola bar, a toffee chew, a meal replacement bar, a spread, a syrup, a powder beverage mix, a cocoa or a chocolate flavored beverage, a pudding, a rice cake, a rice mix, a savory sauce and the like. If desired, the foods may be chocolate or cocoa flavored. Food products may be chocolates and candy bars, such as granola bars, containing nuts, for example, peanuts, walnuts, almonds, and hazelnuts. In foods products (e.g., confectionery, snacks, beverages, dietary supplements) described herein, A-type procyanidin-containing natural sources, for example from peanuts, peanut components e.g., peanut skin, almonds, almond components e.g., almond skin, nut skins, plums (including skin), walnuts, and cranberries may be included in whole/complete (i.e., dried/preserved cranberries) or ground/powdered form. The food is designed to deliver an effective amount of the compounds described herein either by administering the compound individually or in combination.

The compounds for use in the present invention may be of natural origin, for example, derived from a cocoa bean or another natural source known to a person of skill in the art, or prepared synthetically. A person of skill in the art may select natural or synthetic polyphenol based on the use and/or availability or cost.

The compounds may be included in the composition in the form of a cocoa component, for example, chocolate liquor included in chocolate, or may be added independently of cocoa components, for example, as an extract, extract fraction, isolated and purified individual compound, pooled extract fractions or a synthetically prepared compound. The extraction and purification may be conducted as described in U.S. Pat. Nos. 5,554,645 and 6,670,390 to Romanczyk et al., and U.S. Pat. No. 6,627,232 to Hammerstone et al., each of which is hereby incorporated herein by reference.

Cocoa flavanols and/or procyanidins may be provided in the composition of the invention by cocoa ingredients (e.g., chocolate liquor, partially and fully-defatted cocoa solids) containing these compounds or by including chocolate, which may be milk, sweet and semi-sweet, and is preferably dark chocolate, and low fat chocolate. The cocoa ingredients may be prepared using traditional cocoa processing procedures but is preferably prepared using the method described in U.S. Pat. No. 6,015,913 to Kealey et al. Alternatively, to enhance the level of cocoa polyphenols, chocolate liquor and cocoa solids prepared from cocoa beans having a fermentation factor of 275 or less may be used. These ingredients have cocoa polyphenol content that is higher than can be obtained using traditional cocoa processing methods (e.g., with roasting) and fully fermented beans. The chocolate may be prepared using conventional techniques from the ingredients described above or using an improved process for preserving cocoa polyphenols during chocolate manufacturing as described in U.S. Pat. No. 6,312,753 to Kealey et al, and in the International Appl. No. PCT/US99/05414 published as WO99/45788 and in its U.S. counterpart, U.S. Pat. No. 6,194,020, the relevant portions of which are hereby incorporated herein by reference. A chocolate prepared by at least one of the following non-traditional processes is referred to herein as a “chocolate having a conserved amount of cocoa polyphenols”: (i) preparing cocoa ingredients from underfermented or unfermented cocoa beans; (ii) preserving cocoa polyphenol during cocoa ingredient manufacturing process; and (iii) preserving cocoa polyphenol during chocolate manufacturing process. Such non-traditional processes may be used to prepare other cocoa component-containing products (foods e.g., beverages, dietary supplements) designed to contain enhanced levels of flavanols and/or procyanidins.

Synthetic B-type procyanidins may also be used and are prepared by methods known in the art and as described, for example in, U.S. Pat. Nos. 6,420,572; 6,156,912; and 6,864,377, the relevant portions of each of which are hereby incorporated herein by reference.

A-type procyanidins may be of natural origin or synthetically prepared. For example, A-type procyanidins may be isolated from peanut skins as described in Lou et al., Phytochemistry, 51: 297-308 (1999), Karchesy and Hemingway, J. Agric. Food Chem., 34:966-970 (1986), or in U.S. patent application Ser. No. 11/045,648 filed on Jan. 28, 2005 and published as US 2005/0164956, the relevant portions of each being hereby incorporated herein by reference. Mature red peanut skin contain about 17% by weight procyanidins, and among the dimeric procyanidins epicatechin-(4β→8; 2β→O→7)-catechin dominates, with smaller proportion of epicatechin-(4β→8; 2β→O→7)-epicatechin being present. However, in addition to A-type procyanidins having (4→8; 2→O→7) double linkages, procyanidins having (4→6; 2→O→7) double linkages are also found in peanut skins.

Other sources of the A-type procyanidins are cranberries as described, for example in Foo et al., J. Nat. Prod., 63: 1225-1228, and in Prior et al., J. Agricultural Food Chem., 49(3):1270-76 (2001), the relevant portions of each being hereby incorporated herein by reference. Other sources include Ecdysanthera utilis (Lie-Chwen et al., J. Nat. Prod., 65:505-8 (2002)) and Aesculus hippocastanum (U.S. Pat. No. 4,863,956), the relevant portions of each being hereby incorporated herein by reference.

A-type compounds may also be obtained from B-type procyanidins via oxidation using 1,1-diphenyl-2-pycrylhydrazyl (DPPH) radicals under neutral conditions as described in Kondo et al., Tetrahedron Lett., 41: 485 (2000), the relevant portions of which are hereby incorporated herein by reference. Methods of obtaining natural and synthetic B-type procyanidins are well known in the art and are described, for example, in U.S. Pat. Nos. 6,670,390 to Romanczyk et al.; 6,207,842 to Romanczyk et al.; 6,420,572 to Romanczyk et al.; and 6,156,912 to Romanczyk et al., hereby incorporated herein by reference.

A daily effective amount of the compound of the invention may be provided in a single serving in case of a food or a single dosage in case of a pharmaceutical or a dietary supplement or by multiple servings or dosage forms. For example, a confectionery (e.g., chocolate) may contain at least about 100 mg/serving (e.g., 150-200, 200-400 mg/serving). A person of skill in the art will appreciate that the amount effective to achieve the health benefits described herein can be accomplished by administration of a single compound or a mixture of compounds described herein.

The dietary supplement containing the compound(s) of the invention, and optionally another immunity boosting agent, may be prepared using methods known in the art and may comprise, for example, nutrient such as dicalcium phosphate, magnesium stearate, calcium nitrate, vitamins, and minerals.

Further within the scope of the invention is an article of manufacture such as a packaged product comprising the composition of the invention (e.g., a food, a dietary supplement, a pharmaceutical) and a label indicating the presence of, or an enhanced content of the inventive compounds or directing use for boosting immunity, e.g. innate immunity. The packaged product may contain the composition and the instructions for use to boost immunity, e.g. innate immunity. The label and/or instructions for use may refer to any of the methods of use described in this application.

The invention also relates to a method of manufacturing an article of manufacture comprising any of the compositions described herein, packaging the composition to obtain an article of manufacture and instructing, directing or promoting the use of the composition/article of manufacture for any of the uses described herein. Such instructing, directing or promoting includes advertising.

The invention is further described in the following non-limiting examples.

### EXAMPLES

### Example 1

### Materials and Methods

Human Subjects

Forty ml. of peripheral blood was drawn after overnight fasting from 10 healthy male volunteers ranging in age from 22 to 28 years old. Samples were drawn into citrate-containing tubes and mixed 1:1 with Hanks Balanced Salt Solution (HBSS; Invitrogen, Carlsbad, Calif.) without addition of calcium and magnesium. Subjects were asked to refrain from the use of dietary supplements and multi-vitamins for a minimum of 30 days prior to this study. Blood samples were layered over Histopaque-1077 gradient and centrifuged at 500 g for 30 min at room temperature. Peripheral blood mononuclear cells (PBMCs) were then harvested, washed twice in HBSS, and counted using a hemocytometer and an estimation of viability by a trypan blue exclusion assay. Viability was consistently greater than 95%. Cells were finally resuspended in serum free X-VIVO 15 (BioWhittaker, Walkersville, Md.) and numbers were adjusted to 2×10⁶ cells/ml.

Isolation of Monocytes, CD4 and CD8 T-Cells

CD4+ and CD8+ cells were isolated by positive selection. Briefly, 10×10⁷ PBMC cells in 90 μl were incubated with 10 μl of either anti-CD4+ or CD8+ magnetic reagent and incubated at 4° C. for a half hour. The cells were then separated by magnetic cell sorting using a Miltenyi Biotec system, were counted and diluted to a concentration of 2×10⁶ cells/ml with BioWhittaker X Vivo. Autologous plasma from the subject was added to a concentration of 1% by volume. A 96 well plate was treated with antihuman-CD3 in PBS (BD Biosciences) at a concentration of 0.5 μg/ml for 4 hours. After the 4 hour incubation, the plate was washed 4 times with PBS. Purified CD4+ or CD8+ cells were added to each well along with various concentrations of the individual test compounds or controls. The cultures were then incubated at 37° C. for 1 day and supernatants were harvested and stored at −80° C.

For isolation of monocytes from PBMC, the monocyte isolation kit (Miltenyi Biotec) was used, which involves an indirect magnetic labeling system via magnetic depletion of T cells, natural killer cells, B cells, DCs, and basophils. The purity of negatively selected monocytes was assessed by flow cytometry and found to be 95% pure. Cells were resuspended at 2×10⁶ mL in ex vivo 15 medium (Cambrex Bio Science Walkersville, Md.) supplemented with penicillin (50 U/mL), and streptomycin (50 μg/mL). Cells were then dispensed in a volume of 100 μl per well into individual wells of 96-well flat-bottom microtiter plates (Corning, Acton, Mass.). Equal volume of media containing the appropriate test compound and controls was added to triplicate wells of monocyte cultures. Cultures were incubated at 37° C. for 72 hours, pulsed with 1.0 μCi of tritiated thymidine for 12 hours and harvested and counted using a Wallac 1205 Betaplate LCS counter.

Purification of Cocoa Flavanols and B-type Procyanidins by Preparative Normal-Phase HPLC

Approximately 0.7 g of semi-purified cocoa extract (obtained as described in U.S. Pat. No. 5,554,645, hereby incorporated herein by reference) was dissolved in 7 ml of acetone/water/acetic acid in volume ratios of 70:29.5:0.5, respectively. Separations were performed at room temperature using a 5 micron Supelcosil LC-Si 100 Å and the flavanols and/or procyanidins eluted by linear gradient solvent system. The separation of monomers (catechin and epicatechin) and B-type procyanidin oligomers was monitored by UV absorbance at 280 nm and fractions collected at intervals between peaks (corresponding to oligomers). The purity of (−)-epicatechin and (+)-catechin was determined to be 95.6% and 99.85%, respectively.

For certain experiments, fractions with equal retention times from several preparative separations were combined, evaporated under partial vacuum and freeze-dried. Each fraction was resuspended in sterile PBS at a final concentration of 5 mg/ml. All collected fractions representing the monomeric flavanols and the procyanidin oligomers with up to 5 monomeric subunits (i.e., dimers, trimer, tetramers and pentamers) were combined and designated “short-chain flavanol fraction” (SCFF). All fractions containing procyanidin oligomers that contained from 6 to 10 monomeric subunits (heptamers to decamers) were combined and designated “long-chain flavanol fraction” (LCFF).

Preparation of Methylated Epicatechins

Two methylated epicatechins (3′-O-methyl-(−)-epicatechin and 4′-O-methyl-(−)-epicatechin) were also prepared as described below.

HPLC grade methanol, acetonitrile, acetone, isopropanol and acetic acid were purchased from Fischer Scientific (Boston, Mass.). (−) Epicatechin, iodoethane, iodomethane and potassium carbonate were purchased from Aldrich-Sigma Chemical Co. (St. Louis, Mo.). Deuterated NMR solvents (d₄-MeOH, d₆-acetone, d₃-ACN) were purchased from Cambridge Isotope Laboratories (Andover, Mass.) and Aldrich-Sigma Chemical Co.

Anhydrous K₂CO₃ (6.9 g) was magnetically stirred into acetone (250 mL). Epicatechin (2.5 g) was then added and stirred (5-10 min). While stirring, CH₃I or CH₃CH₂I (10 mL) was added slowly. Reaction was carried out at ambient temp in a sealed flask. Reaction was monitored by HPLC-MS in negative ion mode every 2-4 hours until the ratio of epicatechin ([M-1]⁻; m/z 289), 3′-O-Me-epicatechin ([M-1]⁻; m/z 303), and 4′-O-Me-epicatechin ([M-1]⁻; m/z 303) were approximately 1:1:1. The reaction of CH₃CH₂I with epicatechin was monitored in a similar fashion in accordance with expected molecular ions. The crude products were worked up by vacuum filtration of the reaction mixture through a Büchner funnel with a Whatman #4 filter to remove K₂CO₃ solids. Acetone was removed by rotary evaporation under reduced pressure at 40° C. Solids were dissolved in isopropanol then filtered as before to remove any residual K₂CO₃. Solvents were removed by rotary evaporation under reduced pressure at 40° C. to afford a pale brown crusty residue. The synthesis described above was adapted from previously published work (Donovan, L. R., Luthiria, D. L., Stremple, P., Waterhouse, A. L. “Analysis of (+) catechin, (−) epicatechin and their 3′- and 4′-O-methylated analogs, A comparison of sensitive methods.” Journal of Chromatography B, 726 (1999) 277-283.

The purification system consisted of two Agilent 1100 Preparative Pumps (Agilent Technologies, Wilmington, Del.), Agilent 1100 keypad controller, Rheodyne injection valve fitted with a 5 mL loop (Rhonert Park, Calif.), HP1050 UV detector (Hewlett Packard, Palo Alto, Calif.), Luna 10μ Prep C18 (2) 250×50 mm column (Phenomenex, Torrance, Calif.), and a Kipp and Zonen flatbed recorder (Bohemia., NY). Eluents were monitored at 280 nm. Peaks corresponding to compounds of interest were collected, rotary evaporated under reduced pressure at 40° C. to remove organic solvents, then freeze-dried to remove water. Other purification details of epicatechin metabolites are described below.

The crude product mixture of 3′- and 4′-O-Me-epicatechin was purified by gradient elution of B (ACN) into A (0.1% HOAc in H₂O) at 30 mL/min. The gradient was 0-30 min; 28.0-30.0% B, 30-30.01 min; 30.0-50.0% B, 30.01-35 min; 50-100%, 35-40 min; 100-28%, 40-45 min; 28% B.

Analyses of isolated compounds were performed using an Agilent 1100 HPLC coupled to an Agilent 1100 MSD/LC Trap equipped with an API-ES chamber. Compounds were subjected to reverse phase (RP) gradient chromatography over ODS Hypersil 5 microns 100×4.6 mm (Thermo Electron Corp.) at 20 C. The binary solvent system consisted of A (0.1% HOAc in H₂O, v/v) and B (0.1% HOAc in MeOH, v/v). The gradient was 0-20 min; 15-25% B, 20-30 min; 25-50% B, 30-35 min 50-100% B with a flow rate of 1 mL/min. Conditions for the mass spectral analysis in negative ion mode included a capillary voltage of 4000 V, a nebulizing pressure of 40 psi, a drying gas flow of 12 L/min and a temperature of 350° C. Data was collected scanning over a mass range of m/z 120-700 at 3 s/cycle using Agilent ChemStation and Brucker Quant Analysis software. Nuclear magnetic resonance (NMR) spectra were obtained on a Brucker 500 MHz instrument (Brucker, Karlsruhe, Germany). ¹HNMR and ¹³CNMR spectra were recorded in d4-MeOH or d6-acetone.

The purity of 3′-O-methyl-(−)-epicatechin was 99.5% and the purity of 4′-O-methyl-(−)-epicatechin was 99.3%.

Preparation of A-Type Procyanidins

A-type procyanidins were prepared as described in Example 1 of U.S. patent application Ser. No. 11/045,648 filed on Jan. 28, 2005 and published as US 2005/0164956, which is hereby incorporated herein by reference.

Culture Conditions

PBMC or purified T cells were placed in either 96 well round bottom culture plates (2×10⁵ cells/well) or 48 well culture plates (Corning, Corning, N.Y.) at a final concentration of 1×10⁶ cells/well. Cells were cultured in triplicate with the addition of serial dilutions (20 μg/ml, 2.0 μg/ml, or 0.2 μg/ml) of each test compound or a saline control for 16 hours. LPS (100 μg/ml) was then added. Cells for FACS analysis were harvested 2 hours post LPS challenge. Supernatant fractions for cytokine analysis were harvested after 24 hours of LPS challenge.

Cytokines and FACS Analysis

Cytokine profiles were determined using a BD Cytometric Bead Array Human Inflammation KIT (BD Biosciences, San Diego, Calif.). Briefly, 50 μl of culture supernates and standards were added to 100 μl of capture beads and detection reagent. Tubes were incubated for 3 hours at room temperature, washed with 1 ml of wash buffer and centrifuged prior to collection of the supernate. Wash buffer (300 μl) was added and samples immediately analyzed on the flow cytometer after vortexing. Known positive and negative samples and including a standard curve were included with each assay and all samples analyzed in triplicate. In addition, cultured cells (1×10⁶) were washed in PBS with 0.1% bovine serum albumin (PBS/BSA), resuspended in 100 μl PBS/BSA, and incubated with anti-human CD3, CD19, CD69 and CD83 antibody for 1 hour at 4° C. Cells were resuspended in 1.0 ml PBS/BSA and analyzed on the flow cytometer.

Statistical Analysis

Analysis of variance (ANOVA) models were used to compare the induced cytokine levels in each of the 6 treatment conditions (SCFF, LCFF, or control treatment, each with or without LPS challenge). Values not following a normal distribution were transformed using the following methods: square root, logarithmic, non-parametric ranking, and ¼ power transformations when appropriate. Values were then back-transformed. If the results of the overall F test in the analysis of variance were significant, pairwise comparisons were made in order to identify which group differed from the other. All analyses were two-tailed and β values <0.05 were considered statistically significant.

Results

Proliferation and Cytokine Profiles of PBMC

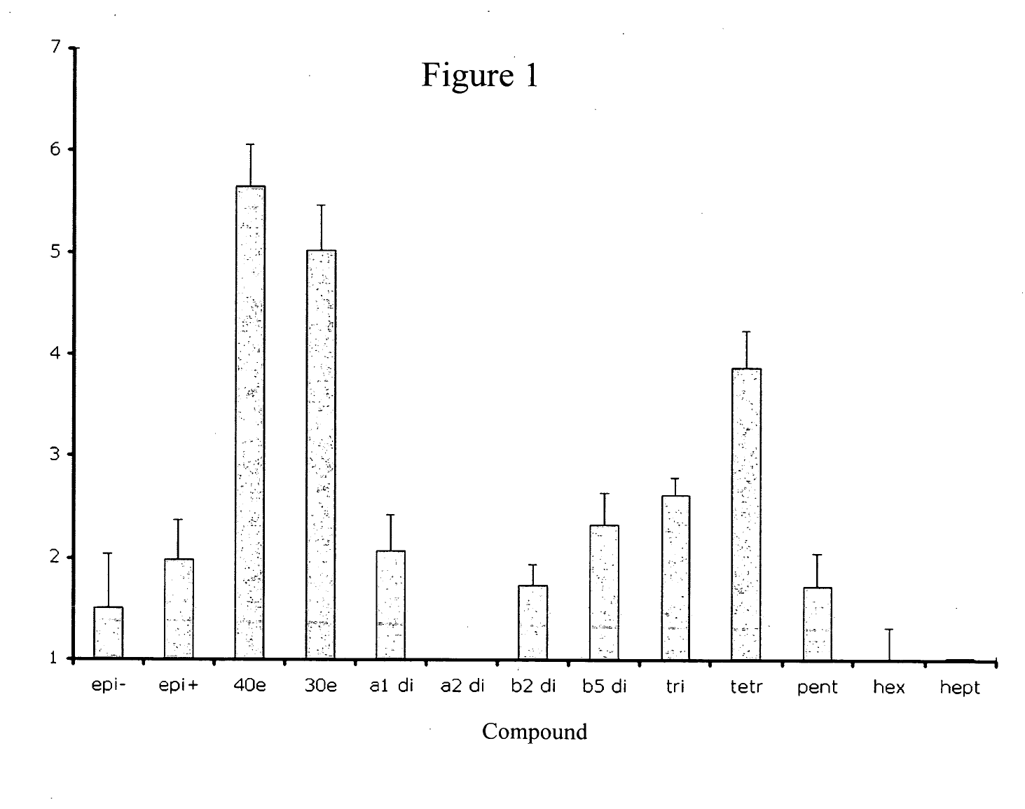

Purified monocyte cultures were treated with individual compounds (including epicatechin, 3′O-methyl-(−)-epicatechin, 4′O-methyl-(−)-epicatechin, B2 dimer, B5 dimer, and the following B-type procyanidin oligomers-trimer, tetramer, pentamer, hexamer and heptamer). O-methylated epicatechin-treated monocytes generated approximately 3 fold the proliferation levels as unmodified epicatechin. As the size of the oligomer increased, from dimer to tetramer, there was a corresponding increase in stimulation. The larger oligomers, pentamer through heptamer demonstrated little or no significant stimulation. (FIG. 1)

In another experiment, supernates from PBMC cultured with different concentrations of SCFF, LCFF, and a medium control, with or without LPS challenge, were analyzed and concentrations of IL-1β, IL-6, IL-10, TNF-α, and GM-CSF measured as described above. (Table 1)

TABLE 1
Cytokine secretion (pg/ml) in the 20 (μg/ml treatment of
LCFF and SCFF in unchallenged and LPS challenged PBMCs.

| | | | Cocoa | | LPS Cocoa |
|---|---|---|---|---|---|
| Cytokine | Oligo | Media | Mean ± SD | Media + LPS | Mean ± SD |
| TNF-α | LCFF | Nd* | Nd | 432 ± 122 | 820.8 ± 100.5*** |
| | SCFF | Nd | Nd | 432 ± 122 | 587.9 ± 136.8*** |
| IL-1β | LCFF | 11.5 ± 4.2 | 36.5 ± 11.8** | 28.4 ± 8.1 | 83.8 ± 19.1** |
| | SCFF | 11.5 ± 4.2 | 23.5 ± 7.5** | 28.4 ± 8.1 | 37.4 ± 6.9** |
| IL-10 | LCFF | 8.9 ± 2.6 | 11.5 ± 2.4 | 66.2 ± 27.7 | 113.4 ± 16.5** |
| | SCFF | 8.9 ± 2.6 | 14.7 ± 5.3 | 66.2 ± 27.7 | 142 ± 62.3** |
| IL-6 | LCFF | 14.7 ± 5.8 | 22.9 ± 10.2 | 720 ± 147 | 1338 ± 224*** |
| | SCFF | 14.7 ± 5.8 | 28.5 ± 5.3** | 720 ± 147 | 795 ± 144.7 |
| GM-CSF | LCFF | 13.6 ± 2.0 | 268.2 ± 134.4** | 85.6 ± 24.6 | 524.1 ± 204.1** |
| | SCFF | 13.6 ± 2.0 | 65.2 ± 25.4** | 85.6 ± 24.6 | 280.6 ± 331 |

*Nd = values were below, or equal to, the detection limit. P values were achieved by using the students T test to compare the Media versus the Cocoa or Media + LPS vs Cocoa + LPS treatments for a given cytokine.
***P < .0001
**P < .05

Treatment of PBMC with 20 μg/ml of LCFF or SCFF alone did not significantly affect the synthesis of TNF-α compared to incubation with medium alone. Both LCFF and SCFF significantly increased the LPS-induced production of TNF-α (432.3±122.2) to 820.8±100.5 pg/ml and 587.9±136.8 pg/ml, respectively (p=0.0001 and p=0.0004, respectively). After incubation with 20 μg/ml of LCFF, IL-1β levels were increased significantly by 36.5±11.8 pg/ml compared to the medium control value of 11.5±4.2 pg/ml and, when activated by the addition of LPS, were 83.8±19.1 pg/ml higher than with LPS alone (28.4±8.1 pg/ml). The SCFF plus LPS treatment had a smaller but still significant increase over LPS media control. In the absence of LPS, levels of IL-6 after treatment of PBMC with LCFF or SCFF were low at all concentrations, and not significantly different from those observed in the medium control. In contrast, following LPS challenge, LCFF treatment was associated with IL-6 levels of 1338±224 pg/ml verses 720±147 LPS control values (p<0.001). Minimal concentrations of IL-10 were detected in the culture supernates of PBMC treated with 20 μg/ml of LCFF or SCFF or medium alone. When combined with LPS, however, treatment with 20 μg/ml LCFF and SCFF resulted in the release of 47.2±24.6 pg/ml and 76.6±53.5 pg/ml of IL-10 over medium control, representing a 71% and 116% increase, respectively, compared to treatment with LPS alone (66.2±27.7 pg/ml; p=0.013 and 0.033, respectively). We note that IL-10 was the only cytokine for which SCFF was a stronger stimulus than LCFF; however, the difference between the two treatments did not reach statistical significance. In the absence of LPS, treatment of PBMC with both SCFF and LCFF resulted in significant induction of GM-CSF compared to medium alone (65.2±25.4 pg/ml and 268.2±134.4 pg/ml, respectively vs. 13.6±2.0 pg/ml). The increase induced by LCFF and SCFF were significantly higher than that seen with the media control. The addition of LPS to test compound treatments resulted in an increase 524±204.1 pg/ml and 280±331 pg/ml, respectively in GM-CSF levels over the values obtained the control LPS media (85.6±24.6). The concentration of GM-CSF seen after incubation with LCFF combined with LPS was significantly increased compared to treatment with LPS alone (p=0.004), whereas the difference between LPS alone and LPS combined with SCFF did not reach statistical significance. Again, the induction of GM-CSF seen in LCFF-treated cells was higher than with SCFF, but the difference did not reach statistical significance.

Dose-dependent increase was observed in the secretion of TNF-α, IL-1, and IL-6 after treatment with LCFF combined with LPS, and in the release of TNF-α and IL-10 after incubation with SCFF plus LPS (data not shown) for the three concentrations studied (0.2, 2.0 and 20 μg/ml). However, significant differences to the results obtained with LPS alone were observed only at the 20 μg/ml concentrations of LCFF and SCFF.

Cytokine Profiles of Treated CD4 and CD8 T Cells

Isolated CD4 and CD8 T cells activated with anti CD3 were treated with (−)-epicatechin, 3′-O-methyl-(−)-epicatechin, B5 dimer and B-type procyanidin hexamer and assayed for cytokine production. Activated CD4 T cells treated with the different cocoa compounds reflected increased levels of cytokine production over media control. 3′-O-methyl-(−)-epicatechin treated CD4 T cells demonstrated significantly higher levels of IFN-γ, TNF-α and IL-10 over media control and (−)-epicatechin treated cells. CD8 T cells in response to (−)-epicatechin and 3′-O-methyl-(−)-epicatechin produced significantly higher amounts of IFN-γ over the media controls.

Activation of Cell Surface Markers

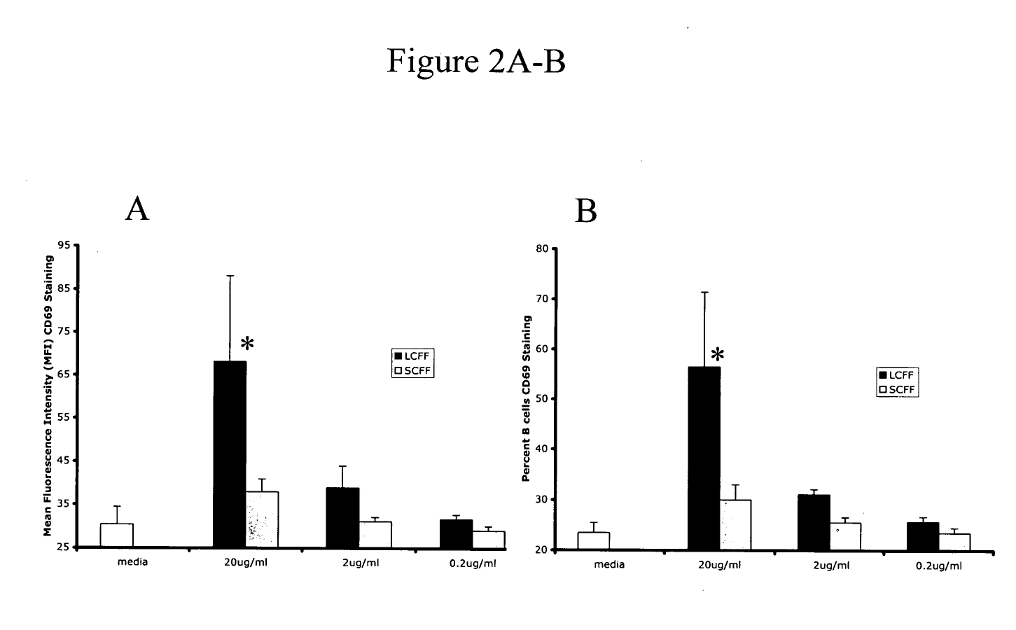

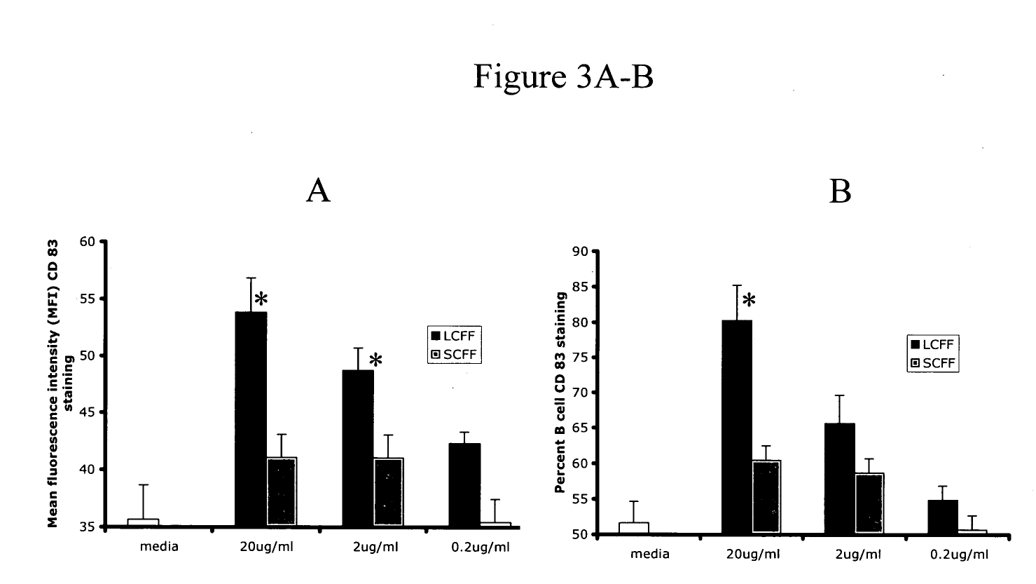

Data on cell surface markers are reported as mean fluorescence intensity (MFI), which is regarded as a measure of receptor density, and percentage of cells expressing the respective activation markers. A significant staining for CD69 after incubation with the cocoa flavonoid fractions was observed on B cells. A dose response analysis was completed for both SCFF and LCFF treatments in LPS challenged and unchallenged cells. LPS treatment did not affect expression of CD 69. Treatment with LCFF was associated with a dose-dependent increase in CD69 expression on B cells, with the concentration of 20 μg/ml giving the highest mean fluorescence intensity (MFI) (FIG. 2A). There was also a greater than two-fold increase in the percentage of CD69+ B cells following LCFF treatment compared with incubation with medium only (57% and 23%, respectively) (FIG. 2B). The MFI and percentage of B cells expressing CD69 was consistently lower after SCFF than after LCFF treatment (FIGS. 2A and B). No statistically significant changes in the intensity of staining or the number of CD69 expressing cells were observed for either of the above treatments after LPS challenge. B cells were the only PBMC subpopulation to demonstrate significant surface expression of CD83 following cocoa treatment. Similar to CD69, CD83 expression was enhanced by LCFF treatment in a dose-dependent manner, with the highest concentration tested (20 μg/ml) resulting in the greatest induction (FIG. 3). In contrast, the MFI for CD83 reached a plateau at a SCFF concentration of 2.0 μg/ml (FIG. 3A). Compared to the medium control, the percentage of CD83+ B cells was significantly elevated in PBMC treated with the highest concentration of LCFF (p=0.049), whereas the increase after incubation with SCFF did not reach statistical significance (52% in control cells, 80% with LCFF, 60% with SCFF) (FIG. 3B). Again, when PBMCs were challenged with LPS, neither of the test treatments caused a significant change in staining profiles.

What is claimed is:

1. A method of boosting innate immunity in a subject in need thereof comprising administering to the subject a composition comprising an effective amount of at least one compound having the formula Aₙ, or a pharmaceutically acceptable salt thereof: 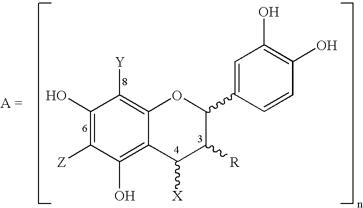
wherein
n is an integer from 2 to 18;
R and X each have either α or β stereochemistry;
R is OH or O-sugar;
the substituents of C-4, C-6 and C-8 are X, Z and Y, respectively, and bonding of monomeric units occurs at C-4, C-6 or C-8;
when any C-4, C-6 or C-8 is not bonded to another monomeric unit, X, Y and Z are hydrogen or a sugar; and
the sugar is optionally substituted with a phenolic moiety at any position, for instance, via an ester bond.

2. The method of claim 1, wherein n is 2-5.

3. The method of claim 2, wherein the subject is a human.

4. The method of claim 3, wherein R is —OH, and when any C-4, C-6 or C-8 is not bonded to another monomeric unit, X, Y and/or Z are hydrogen.

5. The method of claim 4, wherein the subject is a subject having an increased susceptibility to an invader.

6. The method of claim 4, wherein the subject is an immunocompromised subject.

7. The method of claim 4, wherein the subject is a subject suffering from malnutrition.

8. The method of claim 4, wherein the subject is a subject receiving immunosuppressive therapy.

9. The method of claim 4, wherein the subject is a subject having an increased risk of, and/or exposure to, an invader.

10. A method of boosting innate immunity in a subject in need thereof comprising administering to the subject a composition comprising an effective amount of a flavanol selected from the group consisting of epicatechin, catechin, and a pharmaceutically acceptable salt thereof.

11. The method of claim 10, wherein the subject is a human.

12. The method of claim 11, wherein the subject is a subject having an increased susceptibility to an invader.

13. The method of claim 11, wherein the subject is an immunocompromised subject.

14. The method of claim 11, wherein the subject is a subject suffering from malnutrition.

15. The method of claim 11, wherein the subject is a subject receiving immunosuppressive therapy.

16. The method of claim 11, wherein the subject is a subject having an increased risk of, and/or exposure to, an invader.

17. A method of boosting innate immunity in a subject in need thereof comprising administering to the subject a composition comprising an effective amount of a compound which is an oligomer composed of n monomeric, flavan-3-ol units, which flavan-3-ol unit has the following formula: 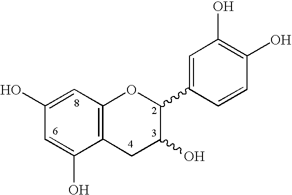
wherein
(i) the monomeric units are connected via interflavan linkages 4→6 and/or 4→8;
(ii) at least two of the monomeric units are additionally linked by an A-type interflavan linkage (4→8; 2→O→7) or (4→6; 2→O→7); and
(iii) n is 2 to 12;
or a pharmaceutically acceptable salt thereof.

18. The method of claim 17, wherein n is 2.

19. The method of claim 18 wherein the subject is a human.

20. The method of claim 19, wherein the subject is a subject having an increased susceptibility to an invader.

21. The method of claim 19, wherein the subject is an immunocompromised subject.

22. The method of claim 19, wherein the subject is a subject suffering from malnutrition.

23. The method of claim 19, wherein the subject is a subject receiving immunosuppressive therapy.

24. The method of claim 19, wherein the subject is a subject having an increased risk of, and/or exposure to, an invader.
